(19) United States
(12) Patent Application Publication
(10) Pub. No.: US 2005/0031696 A1
(43) Pub. Date: Feb. 10, 2005

(54) Oral pharmaceutical formulations of acid-labile active ingredients and process for making same

(75) Inventors: Ujwal Damu Kolhe, Hyderabad (IN); Divi Murali Krishna, Memphis, TN (US); Akhilesh Ashok Dixit, Hyderabad (IN); Abhijit Mukund Deshmukh, Hyderabad (IN); Narayan Daga Rajput, Hyderabad (IN); Mailatur Sivaraman Mohan, Hyderabad (IN); Manne Satyanarayana Reddy, Hyderabad (IN); Muppa Kishore Kumar, Hyderabad (IN); Koilkonda Purender, Hyderabad (IN); Alieti Sanjay Reddy, Hyderabad (IN)

Correspondence Address: DR. REDDY'S LABORATORIES, INC., 200 Somerset Corporate Blvd, SEVENTH FLOOR,, BRIDGEWATER, NJ, 08807-2862, US

(73) Assignees: DR. REDDY'S LABORATORIES LIMITED; DR. REDDY'S LABORATORIES, INC.

(21) Appl. No.: 10/830,486

(22) Filed: Apr. 22, 2004

(30) Foreign Application Priority Data
Apr. 22, 2003 (IN) 340/MAS/2003

(51) Int. Cl. A61K 31/4439; A61K 9/14

(52) U.S. Cl. 424/488; 514/338

(57) ABSTRACT

An oral pharmaceutical formulation in dosage form of acid-labile compounds, methods of treating using the formulation and a process for its production are described.

### CROSS-REFERENCE TO RELATED APPLICATIONS

This application claims priority of Indian Patent Application No. 340/MAS/2003, filed on Apr. 22, 2003, of which entire content is incorporated by reference herein

### BACKGROUND OF THE INVENTION

Certain pharmaceutically active ingredients are acid-labile so as to create several problems in formulating such acid-labile compounds into oral pharmaceutical dosage forms because the acidic environment of the stomach. For example, certain substituted benzimidazole derivatives have poor stability. In particular, they would be rapidly decomposed and colored under moist conditions or in an acidic to neutral aqueous solution. When these compounds are formulated into a preparation for oral administration, they require special measurements to avoid contacts with gastric acid of the stomach. One measurement most commonly used is to coat acid-labile compounds, or its granules or pallets with an enteric coating, which is insoluble in water under acidic conditions and soluble in water under neutral to alkaline conditions. However, the material used in enteric coatings are acidic, which can cause the decomposition of the acid-labile compound. Such decomposition occurs even during the enteric coating process, which results in the coloration of the surface of the core. In order to avoid such problem, an inert subcoating, which is not acidic, is often required between the core and enteric coating, which increase the complexity and the cost of the formulation manufacture process involving acid-labile compounds.

For substances that are labile in acid media, but have better stability in neutral to alkaline media, it is often advantageous to add alkaline reacting inactive constituents in order to increase the stability of the active compound during manufacture and storage. In particular, substituted benzimidazole derivatives such as omeprazole and esomeprazole are not only unstable in acidic condition but also are not stable in neutral solid state. Thus, in order to enhance the storage stability, an alkaline base such as sodium bicarbonate is added to the formulation, and/or the substituted benzimidazole derivatives are converted to their alkaline salts, which are usually more stable than the free species. It is also known that such alkaline base has adverse effects on patients who suffer hypertension, heart failure, etc.

### SUMMARY OF THE INVENTION

In accordance with one aspect, the invention provides a stabilized pre-mix for use in pharmaceutical formulations of acid-labile pharmaceutical active ingredients, which is an admixture of a) an acid-labile pharmaceutical active ingredient; and b) a water-soluble sugar derivative. Preferably, the stabilized premix further includes a pharmaceutically acceptable organic base, which serves as a stabilizer.

In accordance with another aspect, the invention provides an oral pharmaceutical composition in a solid dosage form which includes a) a core containing the stabilized pre-mix, which is free of basic substances; b) an subcoating coated on the core; and c) an enteric coating coated on the subcoating. Preferably, the subcoating is chemically inert.

In accordance with yet another aspect, the invention provides an oral pharmaceutical composition in a solid dosage form that includes a) a core containing the stabilized premix, including the organic base; and b) an enteric coating. Preferably, the core is substantially free of inorganic basic substances. In one embodiment, the enteric coating is coated directly on the core. In another embodiment, which is preferred, the oral pharmaceutical composition further includes a subcoating coated on the core, with the enteric coat is coated on the subcoating.

In yet another aspect, the invention provides a method of inhibiting gastric acid secretion including administering to a mammal in need of such treatment, an effective amount of an oral pharmaceutical compositions described herein.

Yet, in another aspect, the invention provides a process for preparing a stabilized pre-mix for use in pharmaceutical formulations of acid-labile pharmaceutical active ingredients, the process including:
a. dissolving an acid-labile pharmaceutical compound in a ketone solvent;
b. adding a water-soluble sugar derivative to the solution;
c. distilling off the ketone solvent;
d. treating the residue with an aliphatic hydrocarbon solvent until solids separate;
e. isolating said solids thereby obtaining to give said stabilized premix.

Preferably, the process further includes adding an organic base before the ketone solvent is removed.

In yet another aspect, the invention provides a process for preparing a stabilized pre-mix for use in pharmaceutical formulations of acid-labile pharmaceutical active ingredients, the process including:
a) suspending an acid-labile pharmaceutical compound, a water soluble sugar derivative, and an organic base in water or a ketone solvent; and
b) spray-drying the suspension.

Further features of the invention will be apparent from the detailed description herein below set forth.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

The term “pharmaceutical composition” is intended to encompass a product comprising the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

The term “isolating” is used to indicate separation of the compound being isolated regardless of the purity of the isolated compound from any unwanted substance, which presents with the compound as a mixture. Thus, degree of the purity of the isolated or separated compound does not affect the status of “isolating”.

The terms “pharmacologically effective amount”, “pharmaceutically effective dosage”, “pharmaceutically effective amount” or “therapeutically effective amount” mean that amount of a drug or pharmaceutical agent that will elicit the biological or medical response of a tissue, system, animal or human that is being sought by a researcher or clinician.

The term, “acid-labile pharmaceutical compound” means any pharmaceutically active compound, which is not stable in acidic condition or which undergoes degradation or hydrolysis via acid or proton catalyzed reaction and includes substituted benzimidazole derivatives as defined below.

The term, “substituted benzimidazole derivative(s)” mean a compound represented by the following general formula I: 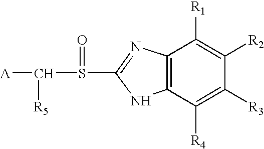
wherein A is an optionally substituted heterocyclic group, R₁, R₂, R₃ and R₄ are the same or different and select from among hydrogen, lower alkyl, lower alkoxy, —CF₃, lower alkylcarbonyloxy, lower alkyloxycarbonyl or halogen and R₅ is H or a lower alkyl group wherein “lower” denotes 1-6 carbon atoms except the compound omerprazole, 5-methoxy-2[[(4-methoxy-3,5 dimethyl-2-pyridinyl)methyl]sulfinyl]-1H-benzimidazole; or the acid labile compound is 2-[(2-dimethylaminobenzyl)sulfinyl]-benzimidazole. The substituted benzimidazole derivative shown above has a chiral center at the sulfur atom and could exist as optically pure or enriched isomers or a racemic mixture. Thus, the term, “substituted benzimidazole derivative(s)” includes each enantiomer, optically enriched isomer, or racemic mixture.

Several substituted benzimidazole derivatives including rabeprazole, omeprazole, esomeprazole, lansoprazole, leminoprazole, pantoprazole and mixtures thereof, are known to be useful for inhibiting gastric acid secretion in mammals and man by controlling gastric acid secretion at the final step of the acid secretory pathway. Thus, in a more general sense, it may be used for prevention and treatment of gastric-acid related diseases in mammals and man, including e.g. reflux esophagitis, gastritis, duodenitis, gastric ulcers and duodenal ulcers. Furthermore, it may be used for treatment of other gastrointestinal disorders where gastric acid inhibitory effect is desirable, e.g. in patients on non-steroidal anti-inflammatory drug (NSAID) therapy, in patients with non ulcer dyspepsia, in patients with symptomatic gastro-esophageal reflux disease, and in patients with gastrinomas. It may also be used in a patient in intensive care situations, in a patient with acute upper gastrointestinal bleeding, pre-and post-operatively to prevent aspiration of gastric acid and to prevent and treat stress ulceration. Further, it may be useful in the treatment of psoriasis as well as in the treatment of Helicobacter infections and diseases related to these, as well as in the treatment or prophylaxis of inflammatory conditions in mammals, including man. However, because these substituted benzimidazole derivatives are not stable at acidic condition and are also sensitive heat, moisture, and light to a certain degree even in neutral condition, multiple layers of pharmaceutical formulations have been developed.

U.S. Pat. Nos. 4,628,098; 4,786,505; 4,853,230; 5,689,333; 5,045,321; 5,093,132; and 5,433,959, of which entire contents are incorporated by reference, teach various stabilizing agents for the disclosed benzimidazole derivatives in the core tablets. These references also show that such compounds are stable in the presence of basic inorganic salts of magnesium, calcium, potassium and sodium. The stability is further consolidated by separating the acid labile benzimidazoles from the acidic components of the enteric coat by an intermediate coating (subcoating).

U.S. Pat. No. 6,013,281, of which entire contents are incorporated by reference, also discloses that a separating layer is formed in situ by direct application of an acidic enteric material on to the alkaline core containing the benzimidazoles (proton pump inhibitors).

However, there are still needs of new formulation technique for acid-labile pharmaceutical compounds.

The core contains an acid-labile pharmaceutical compound premix, which is a mixture or admixture of the acid-labile pharmaceutical compound with a water soluble sugar or sugar derivative such as, for example, sugar alcohols with or without an organic base. The acid-labile pharmaceutical compound premix may be prepared by spray drying suspension of an acid-labile pharmaceutical compound and a water soluble sugar derivative with or without an organic base. Alternatively, the acid-labile pharmaceutical compound premix may also be prepared by Fluid Bed granulation technique, where a solution of an acid-labile pharmaceutical compound with or without an organic base is sprayed on to a water soluble sugar derivative. In one particular variant, the acid-labile pharmaceutical compound premix may be prepared by a process, which a) includes dissolving an acid-labile compound in a ketone solvent; b) adding a water soluble sugar derivative to the solution of step a); c) distilling off the ketone solvent from the mixture formed in step b); d) adding aliphatic hydrocarbon solvents to the residue formed in step c); e) stirring the mixture formed in step d); and f) isolating solids after step e).

After a water soluble sugar derivative is added to the solution of step a), an aliphatic hydrocarbon solvent such as cyclohexane, n-heptane, hexane or mixtures thereof may be added. Furthermore, the solution of step a) can also be purified with charcoal before a water soluble sugar derivative is added.

The ketone solvent includes, for example, acetone, ethyl methyl ketone, methyl isobutyl ketone, diethyl ketone, or mixtures thereof. The water soluble sugar derivatives may be any pharmaceutically acceptable water soluble sugar excipients, preferably having low hydroscopicity and includes, for example, mannitol, lactose, fructose, sorbitol, xylitol, maltodextrin, dextrates, dextrins, lactitol and mixtures thereof. The aliphatic hydrocarbon solvent of step d) includes, for example, cyclohexane, n-heptane, hexane or mixtures thereof. The distillation is preferably done under reduced pressure and preferably at below about 30° C., more preferably at around room temperature.

The isolated solids may be dried under reduce pressure at about 30-35° C. to get water content below 2.0%.

When an oral pharmaceutical composition is prepared with an organic base in accordance with one aspect of the present invention, the organic base may be added the solution of step a) along with a water soluble sugar derivative.

The organic base that may be used in the present invention is a pharmaceutically acceptable organic base, which includes, for example, meglumium, lysine, N,N′-dibenzylethylenediamine, chloroprocain, choline, diethanolamine, ethylenediamine, procaine, and mixtures thereof.

When an organic base is used in the premix and thus in the core, the oral pharmaceutical composition of the present invention does not require an inert subcoating. Also, even when any basic substance is not used in the core, an inert subcoating is not necessarily required in the composition of the present invention to stabilize the acid-labile pharmaceutical compound therein although it may still be beneficial in enhancing the stability of the drug.

The core may also include other pharmaceutically acceptable excipients such as a surfactant, disintergrant, and/or binder. A suitable surfactant includes, for example, one ore more sodium lauryl sulfate, docusate sodium, poloxamer, polyoxyethylene stearates, polyoxyethylene sorbitol esters of fatty acid, and mixtures thereof. The binder may include, for example, Povidone, methylcellulose, hydroxypropyl methylcellulose, ethyl cellulose, hydroxyethyl cellulose, hydroxypropyl cellulose, starch and mixtures thereof. And the disintergrant may includes, for example, crospovidone, croscarmellose sodium, sodium starch glycolate, polacrilline sodium, polacrillin potassium, croscarmellose calcium, low substituted hydroxypropyl cellulose, algenic acid, guar gum, starch, pregelatinised starch, and mixtures thereof.

The core of the present invention may be prepared by homogenously mixing the premix and pharmaceutically acceptable excipients mentioned herein above. The powder mixture is then formulated into small beads, pellets, granules, fine granules, mini-tablets or tablets, hard gelatin or soft gelatin capsules by conventional solid dosage pharmaceutical procedures.

The inert subcoating separates the core from the enteric coating polymer(s) containing free carboxyl groups, which may cause degradation and/or discoloration. The inert subcoating may also serves as a pH-buffering zone in which hydrogen ions diffusing from the outside in towards the alkaline core can react with hydroxyl ions diffusing from the alkaline core towards the surface of the coated articles.

The inert subcoating can be applied to the core pellets or tablets by conventional coating procedures in a suitable coating pan or in fluidized bed apparatus using water and/or conventional organic solvents for the coating solution. In contrast to the available prior art references describing water-soluble or slightly water soluble subcoating in pharmaceutical compositions comprising proton pump inhibitors, the present invention may utilize not only a water insoluble subcoating but also a water soluble subcoating. The water soluble or insoluble polymer that can be used in the inert subcoating includes, for example, sugars, zein, hydroxypropyl cellulose, hydroxypropyl methylcellulose, hydroxyethyl cellulose, polyvinyl alcohol, providone, polyethylene glycol, poloxamer, ethyl cellulose, gelatin, polysine, polyarginine, polyglycine polyvinylpyrolidine, vinyl acetate copolymer and mixtures thereof.

In the case of tablets, the coating may also be applied using the drycoating technique. The inert subcoating may also include pharmaceutically acceptable water-soluble or in water rapidly disintegrating tablet excipients. Ordinary plasticizers, pigments, titanium dioxide talc and other additives may also be included into the inert subcoating. In the case of gelatin capsules the gelatin capsule itself serves as a subcoating. The quantity of the inert subcoating of the present invention may vary from 0.3% to 6%, preferably 0.5 to 4.0%, more preferably 1-3% of the total weight of core.

The enteric coating is applied either directly on to the core or on to the subcoated cores by conventional coating techniques such as, for instance, pan coating or fluidized bed coating using solutions of polymers in water and/or suitable organic solvents or by using latex suspensions of said polymers. Enteric coating polymers that can be used, for example, include hydroxypropyl methylcellulose phthalate, zein, cellulose acetate phthalate, polyvinyl acetate phthalate, methacrylic acid methyl esters/methacrylic acid copolymers, carboxymethylethylcellulose, hydroxypropyl ethylcellulose acetate succinate, acrylic acid polymers and copolymers, and mixtures thereof. The enteric coating can also be applied using water-based polymer dispersions, such as Aquateric® (FMC Corp. Delaware), Eudragit® L 100-55 (Rohm & Haas GmbH, Germany) and Coating CE 5142 (BASF Corp., Delaware). The enteric coating layer can optionally contain a pharmaceutically acceptable plasticizer such as, for instance, cetanol, triacetin, citric acid esters such as, for instance, those known under the trade name Citroflex® (Pfizer, N.Y.) phthalic acid esters, dibutyl succinate or similar plasticizers.

The amount of plasticizer is usually optimized for each enteric coating polymer(s) and is usually in the range of 1-20% of the enteric coating polymer(s). Dispersants such as talc, colorants and pigments may also be included into the enteric coating layer. The weight of enteric coat applied in a said invention is 1-12%, preferably 2-10% and more preferably 4-8% of the weight of core material of the tablet.

In another aspect, the invention also provides methods of treating gastrointestinal inflammatory diseases and gastric acid-related diseases in mammals and man including reflux esophagitis, gastritis, duodenitis, gastric ulcer and duodenal ulcer, using the formulations and pharmaceutical compositions of the present invention. The compounds and compositions of this invention may be administered to a subject in a therapeutically effective amount. In general, the treatment may be determined to alleviate, eliminate, or prevent a given condition based on factors determinable by a skilled physician. By subject is meant a human or an animal. The effective amount (i.e., dosage) of active compound for treatment will vary depending on the route of administration, the condition being treated, its severity, and duration, and the physical state and age of the subject. A skilled physician will monitor the progress of the subject and will adjust the dosage accordingly, depending on whether the goal is to eliminate, alleviate, or prevent a given condition. Generally, the patient's weight, severity of illness, manner of administration and judgment of the prescribing physician should be taken into account in deciding the proper amount. In some cases, it may be necessary to use dosages outside of the stated ranges to treat a patient. Those cases will be apparent to the prescribing physician. Where it is necessary, a physician will also know how and when to interrupt, adjust or terminate treatment in conjunction with a response of a particular patient.

The invention is further defined by reference to the following examples. It will be apparent to those skilled in the art that many modifications, both to the materials and methods, may be practiced without departing from the purpose and interest of the invention. The examples that follow are not intended to limit the scope of the invention as defined hereinabove or as claimed below.

### EXAMPLE 1

### Preparation of Esomeprazole Premix (Meglumine+Mannitol) with 50%

Esomeprazole wet (378.18 grams [W.C. 72.5%] on anhydrous basis 104 grams) was suspended in acetone (520 ml) and stirred for 15-30 minutes to form a clear solution. Charcoal (10.4 grams) was added and stirred for 30-45 minutes. The reaction mass was filtered through hyflow bed and washed with acetone (312 ml). To the filtrate charged meglumine (6.24 grams), mannitol (89.6 grams) and cyclohexane (1.248 liter) was added and then the solvent was distilled under reduced pressure at 20-30° C. Charged cyclohexane (1040 ml) was then added to the residue and distilled under reduced pressure at 20-30° C. Then charged cyclohexane (624 ml) was added and the reaction mass was stirred for 15-30 minutes. The solid was then filtered from the reaction mass and then washed.

The first month stability study of esomeprazole premix, prepared in accordance with the process of Example 1 was conducted at four different conditions. The stability study data is disclosed in Table 1 as well as in FIGS. 1 and 2.

TABLE 1

| | | 50%. Esomeprazole |
|---|---|---|
| | | in meglumine & |
| S. No | Conditions | mannitol mix |
| I. | Long term stability. (25 +/− 2° C.) | Stable |
| | Cold storage stability. (2 to 8° C.) | Stable |

### EXAMPLE 2

Core tablets were prepared by mixing esomeprazole premix with ingredients 2-11 in Table 2 below. The blend was then directly compressed in a tablet compression machine and was further coated with a solution of Zein prepared in 90% of isopropyl alcohol and 10% purified water. The subcoated tablets were then enteric coated with Eudragite® L 100-55 dissolved in isopropyl alcohol. Finally the enteric coated tablets were film-coated with Opadry Pink.

The final product of esomeprazole thus prepared was stored at accelerated stability conditions (40° C. Temp/75% Humidity) for 1 month, 2 months, and 3 months. All samples were analyzed for the presence of compound known to result from the decomposition of esomeprazole (termed as an impurity). The total impurities determined after completion of 3 months was found to be less than 3.0%.

TABLE 2

Strengths

| Sr. No. | Ingredients | 40 mg/Tab | 20 mg/Tab |
|---|---|---|---|
| 1 | Esomeprazole Premix* | 80 | 40 |
| 2 | Magnesium oxide | 20 | 20 |
| 3 | Pearlitol SD 200 | 158.8 | 219.2 |
| 4 | Crospovidone | 22 | 22 |
| 5 | plasdone S-630 | 25 | 21 |
| 6 | Sodium lauryl sulfate | 3.5 | 3.5 |
| 7 | Glycine | 17 | NA |
| 8 | Sodium stearyl fumarate | 10 | 10 |
| 9 | Talc | 3 | 3 |
| 10 | Aerosil | 1 | 1 |
| 11 | Iron Oxide Red | | 0.3 |
| | Total | 340 | 340 |
| | SUB COATING | | |
| 12 | Zein F6000 | 5.1 | 5.1 |
| | ENTERIC COATING | | |
| 13 | Eudragit L100-55 | 17.8373 | 17.8373 |
| 14 | Triethyl Citrate | 1.7837 | 1.7837 |
| 15 | Talc | 0.333 | 0.333 |
| 16 | Titanium Dioxide | 0.4459 | 0.4459 |
| | FILM COATING | | |
| 17 | Opadry Pink OY | 7.48 | 7.48 |
| | Total | 373 | 373 |

*Composition of esomeprazole premix: Esomeprazole 40 mg, mannitol 3 mg and meglumine 37 mg

### EXAMPLE 3

Core tablets were prepared by mixing esomeprazole premix with ingredients 2-11 in Table 3 below. The blend was directly compressed in a tablet compression machine and was further coated with a solution of hydroxypropyl Methyl cellulose (HPMC). The subcoated tablets were then enteric coated with Eudragit® L 100-55 dissolved in isopropyl alcohol. Finally, the enteric coated tablets were film-coated with Opadry Pink.

The final product of esomeprazole thus prepared was stored at accelerated stability conditions (40° C. Temp/75% Humidity) for 1 month, 2 months, and 3 months. All samples were analized for the presence of compound known to result from the decomposition of esomeprazole (termed as an impurity). The total impurities determined after completion of 3 months was found to be less than 3.0%.

TABLE 3

| | | Strengths | |
|---|---|---|---|
| Sr. No. | Ingredients | 40 mg/Tab | 20 mg/Tab |
| 1 | Esomeprazole Premix* | 80 | 40 |
| 2 | Magnesium oxide | 20 | 20 |
| 3 | Pearlitol SD 200 | 158.8 | 219.2 |
| 4 | Crospovidone | 22 | 22 |
| 5 | plasdone S-630 | 25 | 21 |
| 6 | Sodium lauryl sulphate | 3.5 | 3.5 |
| 7 | Glycine | 17 | NA |
| 8 | Sodium stearyl fumarate | 10 | 10 |
| 9 | Talc | 3 | 3 |
| 10 | Aerosil | 1 | 1 |
| 11 | Iron Oxide Red | | 0.3 |
| | Total | 340 | 340 |
| | SUB COATING | | |
| 12 | Hydroxypropyl Methyl cellulose | 13.6 | 13.6 |
| | 5Cps | | |
| 13 | Triethyl citrate | 1.4 | 1.4 |
| | ENTERIC COATING | | |
| 14 | Eudragit L100-55 | 17.8373 | 17.8373 |
| 15 | Triethyl Citrate | 1.7837 | 1.7837 |
| 16 | Talc | 0.333 | 0.333 |
| 17 | Titanium Dioxide | 0.4459 | 0.4459 |
| | FILM COATING | | |
| 18 | Opadry Pink OY | 7.48 | 7.48 |
| | Total | 383 | 383 |

*Composition of esomeprazole premix: Esomeprazole 40 mg, mannitol 3 mg and meglumine 37 mg

### EXAMPLE 4

| | | Quantity in |
|---|---|---|
| S. No | Ingredient | mg/capsule |
| | Core pellet | |
| 1 | Esomeprazole | 40 |
| 2 | Mannitol | 252 |
| 3 | Crospovidone | 18 |
| 4 | Hydroxy propyl methyl cellulose 5 cps | 5 |
| 5 | Sodium lauryl sulphate | 5 |
| 6 | Purified water | q.s |
| | Sub coating | |
| 1 | Hydroxy propyl methyl cellulose 5 cps | 16 |
| 2 | Talc | 0.64 |
| 3 | Titanium dioxide | 0.32 |
| 4 | Purified water | q.s |
| | Enteric coating | |
| 1 | Methacrylic acid copolymer (Type C) | 52 |
| 2 | Triethyl citrate | 5.2 |
| 3 | Talc | 7.75 |
| 4 | Isopropyl alcohol | q.s |

Process of Preparation:
1. Hydroxypropyl methylcellulose 5 cps was dissolved in water.
2. Sodium lauryl sulphate was dissolved in water
3. Esomeprazole, Mannitol and Crospovidone were weighed and passed through mesh #20 and dry blending was done for 5 mins.
4. Sodium lauryl sulphate solution was added to the above blend followed by hydroxypropyl methylcellulose 5 cps solution to make wet mass.
5. Wet mass was passed through Extruder and then to Spheroidizer to make pellets.
6. Wet pellets were dried in a tray direr for 6-7 hrs at 40±5° C.
7. Dried pellets were used for coating purpose.
8. sub coating was performed on core pellets using hydroxypropyl methylcellulose 5 cps. solution containing Talc and Titanium dioxide. The coating was performed in Fluid bed processor or suitable coating machine.
9. Enteric coating was laid over sub coated pellets using methacrylic acid (Type C) solution. This was prepared by dissolving methacrylic acid(Type C) in Isopropyl alcohol and to this solution triethyl citrate and Talc were added.
10. After completion of coating process pellets were cured for 3-4 hrs in a tray drier at 40° C.

### EXAMPLE 5

| S. No | Ingredient | mg/Tablet |
|---|---|---|
| | Core | |
| 1 | Esomeprazole | 40 |
| 2 | Mannitol | 261 |
| 3 | Crospovidone | 25 |
| 4 | Hydroxy propyl methyl cellulose 5 cps | 6 |
| 5 | Sodium lauryl sulphate | 3.5 |
| 6 | Magnesium stearate | 3.5 |
| 7 | Talc | 3.0 |
| 8 | Purified water | q.s |
| | Sub coating | |
| 1 | Hydroxypropyl methyl cellulose 5 cps | 8.75 |
| 2 | Talc | 3.5 |
| 3 | Titanium dioxide | 1.75 |
| 4 | Purified water | q.s |
| | Enteric coating | |
| 1 | Methacrylic acid(Type C) | 14.3 |
| 2 | Triethyl citrate | 1.43 |
| 3 | Talc | 2.2 |
| 4 | Isopropyl alcohol | q.s |

Process of Preparation:
1. Wet mass of blend was prepared in similar to Example 1.
2. The wet mass was dried in tray drier at 40±5° C. for 4-5 hrs.
3. The dried granules were passed through mesh #20
4. Magnesium stearate and Talc were weighed and passed through mesh #40 and blended with dried granules.
5. Final blend was compressed on Rotary tablet compression machine using suitable round shape punches.
6. Core tablets were used for coating purpose.
7. Seal coating was performed on core tablets using Hydroxypropyl methyl cellulose solution containing Talc and Titanium dioxide. The coating was performed in suitable coating instrument.
8. Enteric coating was laid over sub coated pellets using methacrylic acid(Type C) coating solution, which was prepared by dissolving methacrylic acid(Type C) in Isopropyl alcohol and to this solution Triethyl citrate and Talc were added.
9. After completion of coating process tablets were cured for 12 hrs at 40° C.

### EXAMPLE 6

| S. No | Ingredient | mg/cap |
|---|---|---|
| | Core | |
| 1 | Esomeprazole | 40 |
| 2 | Mannitol | 252 |
| 3 | Crospovidone | 18 |
| 4 | Plasdone S-630 | 5 |
| 5 | Sodium lauryl sulphate | 5 |
| 8 | Purified water | q.s |
| | Direct Enteric coating | |
| 1 | Methacrylic acid (Type C) | 53.5 |
| 2 | Triethyl citrate | 5.35 |
| 3 | Talc | 8.0 |
| 4 | Titanium dioxide | 10.72 |
| 5 | Isopropyl alcohol | q.s |

Process of Preparation:
1. Plasdone S-630 was dissolved in water.
2. Sodium lauryl sulphate was dissolved in water separately.
3. Esomeprazole, Mannitol, Crospovidone were weighed and passed through mesh #20 and dry blending was done for 5 mins.
4. Sodium lauryl sulphate solution was added to the above blend followed by plasdone S-630 solution to make wet mass.
5. Wet mass was passed through extruder and then through spheroidizer to make pellets.
6. Wet pellets were dried in a tray drier for 6-7 hrs at 40° C.±5° C.
7. Dried pellets were used for coating purpose.
8. Enteric coating was performed on core pellets using a coating dispersion prepared by dissolving. Methacrylic acid (Type C) and Triethyl citrate in isopropyl alcohol. Talc and Titanium dioxide were suspended in required quantity of isopropyl alcohol and homogenized for 20-30 minutes.
9. Talc & Titanium dioxide dispersion was added to Methacrylic acid solution and mixed for 30 minutes.
10. After completion of coating process pellets were cured for 12 hrs at 40° C.

### EXAMPLE 7

| S. No | Ingredient | mg/Tablet |
|---|---|---|
| 1 | Esomeprazole | 40 |
| 2 | Mannitol | 261 |
| 3 | Crospovidone | 25 |
| 4 | Hydroxypropyl methyl cellulose 5 cps | 6 |
| 5 | Sodium lauryl sulphate | 3.5 |
| 6 | Magnesium. stearate | 3.5 |
| 7 | Talc | 3.0 |
| 8 | Purified water | q.s |
| | Direct Enteric coating | |
| 1 | Methacrylic acid(Type C) | 14.6 |
| 2 | Triethyl citrate | 1.46 |
| 3 | Talc | 2.2 |
| 4 | Titanium dioxide | 2.92 |
| 5 | Isopropyl alcohol | q.s |

Process of Preparation:
1. Wet mass of the blend was prepared similar to Example I.
2. The wet mass was dried in a Tray drier at 40°±5° C. for 4-5 hrs.
3. The dried granules were passed through mesh #20.
4. Magnesium stearate, Talc was weighed and passed through mesh #40 and blended with dried granules.
5. Final blend was compressed on rotary tablet compression machine using suitable round shape punches.
6. Core tablets were used for coating purpose.
7. Direct enteric coating was performed on core tablets using a coating dispersion prepared by dissolving Methacrylic acid and Triethyl citrate in isopropyl alcohol. Talc and Titanium dioxide were suspended in required quantity of isopropyl alcohol and homogenized for 20-30 mins.
8. Talc & Titanium dioxide dispersion was added to methacrylic acid (Type C) solution and mixed for 30 mins.
9. After completion of coating process pellets were cured for 12 hrs at 40° C.

### EXAMPLE 8

| S. No | Ingredient | mg/cap |
|---|---|---|
| 1 | Esomeprazole | 40 |
| 2 | Meglumine | 3 |
| 3 | Mannitol | 33.5 |
| 4 | Pearlitol SD200 | 211 |
| 5 | Crospovidone | 22 |
| 6 | Sodium lauryl sulphate | 3.5 |
| 7 | Plasdone S-630 | 21 |
| 8 | Talc | 3.0 |
| 9 | Mg stearate | 5.0 |

Process of Preparation:
1. Ingredients from 1 to 5 and 7 were weighed and passed through mesh #20.
2. Sodium lauryl sulphate was passed through mesh #20 and added to the above blend in gradient manner.
3. Blend obtained from step 2 was mixed in double cone blender for 10 minutes.
4. Ingredients 8 and 9 were weighed and passed through mesh #40 and added to the double cone blender and lubricated for 5 minutes.
5. Final blend was compressed on tablet compression machine using suitable round shape punches.
6. Core tablets were used for coating purpose.
7. Direct enteric coating was performed similar to Example 4.

### EXAMPLE 9

| S. No | Ingredient | mg/cap |
|---|---|---|
| 1 | Esomeprazole | 40 |
| 2 | Meglumine | 3.0 |
| 3 | Mannitol | 239 |
| 4 | Crospovidone | 18 |
| 5 | Hydroxypropyl methyl cellulose 5 cps | 5.0 |
| 6 | Sodium lauryl sulphate | 5.0 |
| 7 | Purified water | q.s |

Process of Preparation:
1. Hydroxypropyl methylcellulose 5 cps was dissolved in water.
2. Sodium lauryl sulphate was dissolved in water
3. Esomeprazole, meglumine, mannitol, Crosspovidone were weighed and passed through mesh #20 and dry blending was done for 5 minutes.
4. Sodium lauryl sulphate solution was added to the above blend followed by hydroxy-propyl methylcellulose solution to make a wet mass.
5. Wet mass was passed through extruder and then to spheroidizer to make pellets.
6. Wet pellets were dried in a tray drier for 6-7 hrs at 40° C.±5° C.
7. Dried pellets were used for coating purpose.
8. Enteric coating was performed similar Example 3.

### EXAMPLE 10

Process of Preparation:

1. 30/80 fractions of granules obtained from step No. 4 of Example 2 were taken and filled in to suitable hard gelatin capsules and used for coating purpose.
2. Seal and enteric coating were performed similar to Example 2.

### EXAMPLE 11

Process of Preparation:

1. Mesh 30/80 fractions of granules obtained from step No. 4 of Example 3 were taken and filled in suitable hard gelatin capsules and used for coating purpose.
2. Enteric coating was performed as like in Example 3.

### EXAMPLE 12

| S. No | Ingredient | mg/Tablet |
|---|---|---|
| | First layer | |
| 1 | Zein | 3.5 |
| 2 | Triethyl citrate | 0.35 |
| 3 | Talc | 0.87 |
| 4 | Acetone + water (80:20) | q.s |
| | Second layer | |
| 1 | Eudragit L-100 55 | 14.30 |
| 2 | Triethyl citrate | 1.43 |
| 3 | Talc | 2.2 |
| 4 | Isopropyl alcohol | q.s |

Process of Preparation:
1. Core tablets were prepared as like in Formula-II and were coated with following coating composition.
2. First layer was performed on core tablets with zein solution prepared by dissolving zein in required quantity of Acetone & water (80:20). Triethyl citrate & Talc were added to above solution and stirred for 15 mins.
3. After completion of First layer second layer was coated similar to Example 2.

### EXAMPLE 13

Process of Preparation:

1. Core tablets were prepared similar to Example 5.
2. Multiple layer coatings were performed as like in Example 9.

### EXAMPLE 14

| S. No | Ingredient | mg/cap |
|---|---|---|
| 1 | Hydroxy propyl methyl cellulose pthalate HS-55 | 25 |
| 2 | Triethyl citrate | 2.5 |
| 3 | Talc | 7.0 |
| 4 | Acetone + IPA solution (1:1) | q.s |

Process of Preparation:

Core tablets were prepared similar to Example 2 and were seal coated as like in Example 2. Enteric coating was performed using above formula.

### EXAMPLE 15

| | | Strengths | |
|---|---|---|---|
| Sr. No. | Ingredients | 40 mg/Tab | 20 mg/Tab |
| 1 | Esomeprazole Premix* | 80 | 40 |
| 2 | Magnesium oxide | 20 | 20 |
| 3 | Pearlitol SD 200 | 158.8 | 219.2 |
| 4 | Cross Povidone | 22 | 22 |
| 5 | plasdone S-630 | 25 | 21 |
| 6 | Sodium lauryl sulphate | 3.5 | 3.5 |
| 7 | Glycine | 17 | NA |
| 8 | Sodium stearyl fumarate | 10 | 10 |
| 9 | Talc | 3 | 3 |
| 10 | Aerosil | 1 | 1 |
| 11 | Iron Oxide Red | | 0.3 |
| | Total | 340 | 340 |
| | SUB COATING | | |
| 12 | Hydroxypropyl Methyl cellulose 5 Cps | 13.6 | 13.6 |
| 13 | Triethyl citrate | 1.4 | 1.4 |
| | ENTERIC COATING | | |
| 14 | Eudragit L100-55 | 17.8373 | 17.8373 |
| 15 | Triethyl Citrate | 1.7837 | 1.7837 |
| 16 | Talc | 0.333 | 0.333 |
| 17 | Titanium Dioxide | 0.4459 | 0.4459 |
| | FILM COATING | | |
| 18 | Opadry Pink OY | 7.48 | 7.48 |

*Composition of esomeprazole premix: Esomeprazole 40 mg, mannitol 3 mg and meglumine 37 mg

Process:

Core of tablet was prepared by mixing esomeprazole premix with all ingredients from 2 to 11, further blend was directly compressed over tablet compression machine, it was further coated with solution of HPMC. Subcoated tablets were then enteric coated with Eudragit L 100-55 dissolved in isopropyl alcohol. Finally enteric coated tablets were film coated Opadry Pink.

The final product of esomeprazole thus prepared was stored at accelerated stability conditions (40° C. Temp/75% Humidity) for 1 month, 2 months, and 3 months. All samples were analysed for the presence of compound known to result from the decomposition of esomeprazole (termed as an impurity). The total impurity determined after completion of 3 months was found to be less than 3.0%.

Although the invention has been described in a preferred form with a certain degree of particularity, it is understood that the present disclosure of the preferred form has been made only by way of example, and that numerous changes in the details of construction and combination and arrangement of procedures and parts may be made without departing from the spirit and scope of the invention as hereinafter claimed. It is intended that the patent shall cover by suitable expression in the appended claims, whatever features of patentable novelty exist in the invention disclosed.

1. A stabilized pre-mix for use in pharmaceutical formulations of acid-labile pharmaceutical active ingredients, said pre-mix comprising an admixture of
a) an acid-labile pharmaceutical active ingredient; and
b) a water-soluble sugar derivative.

2. The stabilized premix of claim 1, further comprising pharmaceutically acceptable organic base.

3. The stabilized pre-mix of claim 2, wherein said pharmaceutically acceptable organic base is selected from the group consisting of meglumine, lysine, N,N′-dibenzylethylenediamine, chloroprocain, choline, diethanolamine, ethylenediamine, procaine, and mixtures thereof.

4. The stabilized pre-mix of claim 1, wherein said acid-labile pharmaceutical active ingredient is a substituted benzimidazole derivative having the general formula I: 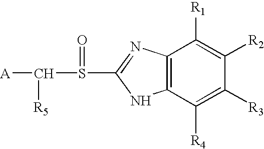
wherein A is an optionally substituted heterocyclic group, R₁, R₂, R₃ and R4 are the same or different and select from among hydrogen, lower alkyl, lower alkoxy, —CF₃, lower alkylcarbonyloxy, lower alkyloxycarbonyl or halogen and R₅ is H or a lower alkyl group wherein “lower” denotes 1-6 carbon atoms except the compound omerprazole, 5-methoxy-2[[(4-methoxy-3,5 dimethyl-2-pyridinyl)methyl]sulfinyl]-1H-benzimidazole; or the acid labile compound is 2-[(2-dimethylaminobenzyl)sulfinyl]-benzimidazole.

5. The stabilized pre-mix of claim 4, wherein said substituted benzimidazole derivative is in a free species form.

6. The stabilized pre-mix of claim 5, wherein said substituted benzimidazole derivative is selected from the group consisting of rabeprazole, omeprazole, esomeprazole, lansoprazole, leminoprazole, pantoprazole and mixtures thereof.

7. The stabilized pre-mix of claim 1, wherein said water soluble sugar derivative is selected from group consisting of mannitol, lactose, fructose, sorbitol, xylitol, maltodextrin, dextrates, dextrins, lactitol and mixtures thereof.

8. The stabilized pre-mix of claim 3, wherein said acid-labile pharmaceutical compound is esomeprazole.

9. The stabilized pre-mix of claim 8, wherein said water-soluble sugar derivative is mannitol.

10. The stabilized pre-mix of claim 9, wherein said pharmaceutically acceptable organic base is meglumine.

11. An oral pharmaceutical composition in a solid dosage form comprising:
b) a core comprising the stabilized pre-mix of claim 1, which is free of basic substances;
c) an subcoating coated on the core; and
d) an enteric coating coated on the subcoating.

12. The oral pharmaceutical composition of claim 11, wherein said subcoating is chemically inert.

13. The oral pharmaceutical composition of claim 11, wherein said acid-labile pharmaceutical compound is a substituted benzimidazole derivative having the general formula I: 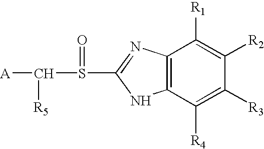
wherein A is an optionally substituted heterocyclic group, R1, R₂, R₃ and R4 are the same or different and select from among hydrogen, lower alkyl, lower alkoxy, —CF₃, lower alkylcarbonyloxy, lower alkyloxycarbonyl or halogen and R₅ is H or a lower alkyl group wherein “lower” denotes 1-6 carbon atoms except the compound omerprazole, 5-methoxy-2[[(4-methoxy-3,5 dimethyl-2-pyridinyl)methyl]sulfinyl]-1H-benzimidazole; or the acid labile compound is 2-[(2-dimethylaminobenzyl)sulfinyl]-benzimidazole.

14. The oral pharmaceutical composition of claim 13, wherein said substituted benzimidazole derivative is in a free species form.

15. The oral pharmaceutical composition of claim 13, wherein said substituted benzimidazole derivative is selected from the group consisting of rabeprazole, omeprazole, esomeprazole, lansoprazole, leminoprazole, pantoprazole and mixtures thereof.

16. The oral pharmaceutical composition of claim 11, wherein said acid-labile pharmaceutical compound is esomeprazole.

17. The oral pharmaceutical composition of claim 11, wherein said water-soluble sugar derivative is selected from mannitol, lactose, fructose, sorbitol, xylitol, maltodextrin, dextrates, dextrins, lactitol and mixtures thereof.

18. The oral pharmaceutical composition of claim 17, wherein said water-soluble sugar derivative is mannitol.

19. The oral pharmaceutical composition of claim 11, wherein said subcoating layer comprises one or more water soluble or insoluble polymer substances selected from the group consisting of sugars, hydroxypropyl cellulose, hydroxypropyl methylcellulose, hydroxyethyl cellulose, polyvinyl alcohol, providone, polyethylene glycol, poloxamer, ethyl cellulose, gelatin, zein, polysine, polyarginine, polyglycine polyvinylpyrolidine, vinyl acetate copolymer and mixtures thereof.

20. The oral pharmaceutical composition of claim 11, wherein said enteric coating comprises a polymer selected from the group consisting of hydroxypropyl methylcellulose phthalate, zein, cellulose acetate phthalate, polyvinyl acetate phthalate, methacrylic acid methyl esters/methacrylic acid copolymers, carboxymethylethylcellulose, hydroxypropyl ethylcellulose acetate succinate, acrylic acid polymers and copolymers, and mixtures thereof.

21. The oral pharmaceutical composition of claim 11, which further comprises a pharmaceutically acceptable surfactant.

22. The oral pharmaceutical composition of claim 21, wherein said pharmaceutically acceptable surfactant is selected from the group consisting of sodium lauryl sulfate, docusate sodium, poloxamer, polyoxyethylene stearates, polyoxyethylene sorbitol esters of fatty acid, and mixtures thereof.

23. The oral pharmaceutical composition of claim 11, wherein said stabilized premix includes 30-60% of the acid-labile pharmaceutical compound and 30-60% of the sugar alcohol, by total weight of the premix.

24. An oral pharmaceutical composition in a solid dosage form comprising:
a. a core comprising the stabilized premix of claim 2; and
b. an enteric coating.

25. The oral pharmaceutical composition of claim 24, wherein said core is substantially free of inorganic basic substances.

26. The oral pharmaceutical composition of claim 24, wherein said enteric coating is coated directly on the core.

27. The oral pharmaceutical composition of claim 24, further comprising a subcoating coated on said core, wherein said enteric coat is coated on said subcoating.

28. The oral pharmaceutical composition of claim 27, wherein said subcoating comprises one or more water soluble or insoluble polymer substances selected from the group consisting of sugars, hydroxypropyl cellulose, hydroxypropyl methylcellulose, hydroxyethyl cellulose, polyvinyl alcohol, providone, polyethylene glycol, poloxamer, ethyl cellulose, gelatin, zein, polysine, polyarginine, polyglycine polyvinylpyrolidine, vinyl acetate copolymer and mixtures thereof.

29. The oral pharmaceutical composition of claim 24, wherein said enteric coating comprises a polymer selected from the group consisting of hydroxypropyl methylcellulose phthalate, zein, cellulose acetate phthalate, polyvinyl acetate phthalate, methacrylic acid methyl esters/methacrylic acid copolymers, carboxymethylethylcellulose, hydroxypropyl ethylcellulose acetate succinate, acrylic acid polymers and copolymers, and mixtures thereof.

30. The oral pharmaceutical composition of claim 24, wherein said acid-labile pharmaceutical compound is a substituted benzimidazole derivative having the general formula I: 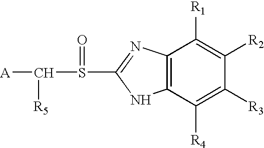
wherein A is an optionally substituted heterocyclic group, R₁, R₂, R₃ and R4 are the same or different and select from among hydrogen, lower alkyl, lower alkoxy, —CF₃, lower alkylcarbonyloxy, lower alkyloxycarbonyl or halogen and R₅ is H or a lower alkyl group wherein “lower” denotes 1-6 carbon atoms except the compound omerprazole, 5-methoxy-2[[(4-methoxy-3,5 dimethyl-2-pyridinyl)methyl]sulfinyl]-1H-benzimidazole; or the acid labile compound is 2-[(2-dimethylaminobenzyl)sulfinyl]-benzimidazole.

31. The oral pharmaceutical composition of claim 30, wherein said substituted benzimidazole derivative is in a free species form.

32. The oral pharmaceutical composition of claim 24, wherein said substituted benzimidazole derivative is selected from the group consisting of rabeprazole, omeprazole, esomeprazole, lansoprazole, leminoprazole, pantoprazole and mixtures thereof.

33. The oral pharmaceutical composition of claim 24, wherein said pharmaceutically acceptable organic base is selected from the group consisting of meglumine, lysine, N,N′-dibenzylethylenediamine, chloroprocain, choline, diethanolamine, ethylenediamine, procaine, and mixtures thereof.

34. The oral pharmaceutical composition of claim 24, wherein said water soluble sugar derivative is selected from mannitol, lactose, fructose, sorbitol, xylitol, maltodextrin, dextrates, dextrins, lactitol and mixtures thereof.

35. The oral pharmaceutical composition of claim 27, wherein said acid-labile pharmaceutical compound is esomeprazole.

36. The oral pharmaceutical composition of claim 35, wherein said water-soluble sugar derivative is mannitol.

37. The oral pharmaceutical composition of claim 35, wherein said pharmaceutically acceptable organic base is meglumine.

38. The oral pharmaceutical composition of claim 24, which further comprises a pharmaceutically acceptable surfactant.

39. The oral pharmaceutical composition of claim 33, wherein said pharmaceutically acceptable surfactant is selected from the group consisting of sodium lauryl sulfate, docusate sodium, poloxamer, polyoxyethylene stearates, polyoxyethylene sorbitol esters of fatty acid, and mixtures thereof.

40. A method of inhibiting gastric acid secretion comprising administering to a mammal in need of treatment, an effective amount of an oral pharmaceutical composition of claim 24.

41. A method of inhibiting gastric acid secretion comprising administering to a mammal in need of treatment, an effective amount of an oral pharmaceutical composition of claim 11.

42. A process for preparing a stabilized pre-mix for use in pharmaceutical formulations of acid-labile pharmaceutical active ingredients, said process comprising:
a. dissolving an acid-labile pharmaceutical compound in a ketone solvent;
b. adding a water-soluble sugar derivative to the solution;
c. distilling off the ketone solvent;
d. treating the residue with an aliphatic hydrocarbon solvent until solids separate;
e. isolating said solids thereby obtaining to give said stabilized premix.

43. The process of claim 42, further comprising adding an organic base before the ketone solvent is removed.

44. A process for preparing a stabilized pre-mix for use in pharmaceutical formulations of acid-labile pharmaceutical active ingredients, said process comprising:
b) suspending an acid-labile pharmaceutical compound, a water soluble sugar derivative, and an organic base in water or a ketone solvent; and
a. b) spray-drying the suspension.

45. An oral pharmaceutical composition in a solid dosage form, which is prepared according to the process of claim 43.

46. An oral pharmaceutical composition in a solid dosage form, which is prepared according to the process of claim 44.
